(19) United States
(12) Patent Application Publication
(10) Pub. No.: US 2023/0277436 A1
(43) Pub. Date: Sep. 7, 2023

(54) HAIR CONDITIONING COMPOSITION FOR IMPROVED DEPOSITION

(71) Applicant: Conopco, Inc., d/b/a UNILEVER, Englewood Cliffs, NJ (US)

(72) Inventors: Richard Jonathan BARFOOT, Chesterfield (GB); Michael James COOKE, Wirral (GB); Cesar Ernesto MENDOZA FERNANDEZ, Liverpool (GB); Paul Damien PRICE, Bebington, Wirral (GB)

(21) Appl. No.: 18/010,314

(22) PCT Filed: Jun. 15, 2021
(86) PCT No.: PCT/EP2021/066151

§ 371 Date: Dec. 14, 2022

(30) Foreign Application Priority Data
Jun. 19, 2020 (EP) 20181258.3

(51) Int. Cl. A61K 8/898; A61K 8/41; A61K 8/04; A61K 8/891; A61Q 5/12; A61K 8/34

(52) U.S. Cl. CPC A61K 8/898; A61K 8/416; A61K 8/042; A61K 8/891; A61Q 5/12; A61K 8/342; A61K 2800/5922; A61K 2800/596

(57) ABSTRACT

Improved particulate benefit agent deposition onto hair is achieved with a corn position comprising: (i) a linear, cationic conditioning surfactant; (ii) a linear fatty material; (iii) a particulate benefit agent (iv) a branched cationic co-surfactant, selected from structure 1, wherein the molar ratio of branched cationic co-surfactant (iv) to linear cationic surfactant (i) is in the range of from 1:20 to 1:1.

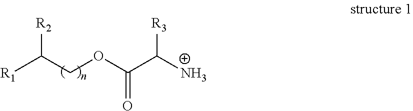

### FIELD OF THE INVENTION

The invention is concerned with conditioning compositions for the treatment of hair, containing a branched co-surfactant and a benefit agent to be deposited onto the hair during use and particularly relates to a conditioning composition that enables increased amounts of benefit agent to be deposited.

### BACKGROUND AND PRIOR ART

In personal care compositions, such as hair treatment compositions, the deposition and delivery of benefit agents are often key drivers of product performance. For example, many of the hair conditioner products in the market today work to deliver benefits to hair by depositing benefit agents such as fragrance materials, silicones and damage repair actives onto the hair during the wash and care process.

However, consumers report being disappointed by the level of benefit derived from use of some compositions. This is usually caused by insufficient amount of benefit agents being delivered to the surface. It is, therefore, desirable to develop compositions that provide improved delivery of benefit materials to a surface, for example hair.

Various types of branched cationic compounds are known in hair treatment compositions for a variety of benefits.

WO 17/172117 discloses a composition for treating keratinous substrates comprising a cationic agent comprising a defined first quaternary ammonium compound and an imidazoline compound, a modified starch, two silane compounds, a cationic vinylpyrrolidone polymer and water. Hair treated with the compositions is purported to have improved mass, body, volume, to be easily rinsed, to dry fast, to stay clean longer and be sufficiently conditioned. US 2005/175569 discloses cosmetic compositions, for example for conditioning and styling hair, comprising a cationic surfactant, which may be a quaternary ammonium salt.

JP 2005-060271 discloses an aqueous hair cosmetic composition that can comprise (A) a dimethylpolysiloxane represented by general formula (1), (B) a dimethylpolysiloxane represented by general formula (2), (C) a cyclic dimethylpolysiloxane represented by general formula (3) at a ratio of [(B)+(C)]/(A) greater than or equal to 1; and (D) an additional quaternary ammonium component. The composition is said to provide a range of conditioning benefits to hair in the wet, rinse and dry stages.

Our own published applications WO 02/102334 and WO 01/43718 provide aqueous hair treatment compositions having cleansing and conditioning properties that comprise quaternary ammonium based cationic surfactants having defined hydrocarbyl chains.

Whilst branched materials are known in home and personal care products, they have not been applied effectively to provide improved deposition of benefit agents onto hair.

Product rheology is a key attribute to consumers. We have, however, found that adding branched surfactant materials into gel networks disrupts the gel structure and consequently reduces viscosity and yield stress to unacceptably low levels.

Despite the prior art, there remains a need to deliver improved delivery of benefits to hair without compromising on consumer desired viscosity characteristics. Consumers strongly prefer thicker products as they associate this with efficacy and quality. However, if it is too thick, pouring from a bottle can become difficult.

Experienced formulators in the field normally compensate for reduced viscosity by adding viscosity modifiers such as polymeric thickeners. However, this brings on other problems such as processing complications, lumpy appearance (the so-called “fish eye” appearance) as well as environmental and cost impact.

We have now surprisingly found that compositions comprising a combination of certain branched co-surfactants in combination with linear conditioning surfactant and used at a specific ratio, provide an unexpectedly large enhancement in the deposition of benefit agents (for example silicone) whilst maintaining excellent product rheology, particularly viscosity and yield stress.

All percentages quoted herein are by weight based on total weight, unless otherwise stated. All amounts quoted herein are based on 100% activity of materials, unless otherwise stated.

### Definition of the Invention

Accordingly, there is provided a conditioning composition comprising:

A composition comprising:
(i) 0.01 to 10 wt % of a linear, cationic conditioning surfactant;
(ii) 0.1 to 10 wt % of a linear fatty material;
(iii) a particulate benefit agent selected from conditioning actives and mixtures thereof;
(iv) 0.01 to 5 wt %, at 100% active, of a branched cationic co-surfactant, selected from structure 1,

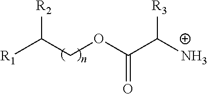

wherein:
R₁ and R₂ comprise linear alkyl chains, saturated or unsaturated, with carbon-carbon chain lengths of from C₄ to C₂₀;
R₃ comprise either a proton or linear or branched alkyl chains, saturated or unsaturated, with carbon-carbon chain lengths of from C₁ to C₅;
n has a range of from 0 to 10;
X is an organic or inorganic anion;

wherein the molar ratio of branched cationic co-surfactant (iv) to linear cationic surfactant (i) is in the range of from 1:20 to 1:1.

In a second aspect, the invention provides a method of increasing deposition of a particulate benefit agent selected from conditioning actives, preferably silicone emulsion and mixtures thereof to hair comprising the step of applying to hair a conditioning composition of the first aspect.

The method of the invention preferably comprises an additional step of rinsing the composition from the hair.

Preferably, the method is a method of increasing silicone deposition to hair comprising the steps of applying to hair a composition as defined by the first aspect of the invention and rinsing the hair with water.

Compositions in accordance with the invention are preferably formulated as conditioners for the treatment of hair (typically after shampooing) and subsequent rinsing.

The conditioning compositions of the invention are not cleansing compositions and, as such, do not comprise anionic cleansing surfactants, for example sodium lauryl ether sulphate.

### General Description of the Invention

Preferably, the treatment composition is selected from a rinse-off hair conditioner, a hair mask, a leave-on conditioner composition, and a pre-treatment composition, more preferably selected from a rinse-off hair conditioner, a hair mask, a leave-on conditioner composition, and a pre-treatment composition, for example an oil treatment, and most preferably selected from a rinse-off hair conditioner, a hair mask and a leave-on conditioner composition. The treatment composition is preferably selected from a rinse-off hair conditioner and a leave-on conditioner.

Rinse off conditioners for use in the invention are conditioners that are typically left on wet hair for 1 to 2 minutes before being rinsed off.

Hair masks for use in the present invention are treatments that are typically left on the hair for 3 to 10 minutes, preferably from 3 to 5 minutes, more preferably 4 to 5 minutes, before being rinsed off.

Leave-on conditioners for use in the invention are typically applied to the hair and left on the hair for more than 10 minutes, and preferably are applied to the hair after washing and not rinsed out until the next wash.

The Linear Cationic Conditioning Surfactant (i)

Conditioner compositions will comprise a linear cationic conditioning surfactant, which is cosmetically acceptable and suitable for topical application to the hair.

Preferably, the linear cationic conditioning surfactants have the formula 1: N⁺(R¹)(R²)(R³)(R⁴), wherein R¹, R², R³ and R⁴ are independently (C₁ to C₆) alkyl or benzyl.

In formula 1, preferably, one, two or three of R¹, R², R³ and R⁴ are independently (C₄ to C₃₀) alkyl and the other R¹, R², R³ and R⁴ group or groups are (C₁-C₆) alkyl or benzyl.

More preferably, one or two of R¹, R², R³ and R⁴ are independently (C₆ to C₃₀) alkyl and the other R¹, R², R³ and R⁴ groups are (C₁-C₆) alkyl or benzyl groups. Optionally, the alkyl groups may comprise one or more ester (—OCO— or —COO—), amido (—NOC— or NCO—), and/or ether (—O—) linkages within the alkyl chain. Alkyl groups may optionally be substituted with one or more hydroxyl groups. Alkyl groups may be straight chain or branched and, for alkyl groups having 3 or more carbon atoms, cyclic. The alkyl groups may be saturated or may contain one or more carbon-carbon double bonds (e.g., oleyl). Alkyl groups are optionally ethoxylated on the alkyl chain with one or more ethyleneoxy groups.

Suitable quaternary amine salts for use in conditioner compositions according to the invention are quaternary amine salt comprising from 12 to 24 carbon atoms, preferably from 16 to 22 carbon atoms.

Suitable quaternary amine salts for use in conditioner compositions according to the invention include cetyltrimethylammonium chloride, behenyltrimethylammonium chloride, Behentrimonium methosulphate, BehenylAmido Propyl Di-Methyl Amine, cetyltrimethylammonium chloride, cetylpyridinium chloride, tetramethylammonium chloride, tetraethylammonium chloride, octyltrimethylammonium chloride, dodecyltrimethylammonium chloride, hexadecyltrimethylammonium chloride, octyldimethylbenzylammonium chloride, decyldimethylbenzylammonium chloride, stearyldimethylbenzylammonium chloride, Stearalkonium Chloride, Stearalkonium methosulphate, didodecyldimethylammonium chloride, dioctadecyldimethylammonium chloride, tallowtrimethylammonium chloride.

dihydrogenated tallow dimethyl ammonium chloride (e.g., Arquad 2HT/75 from Akzo Nobel) and cocotrimethylammonium chloride.

Preferred quaternary amine salts selected from behenyltrimethylammonium chloride, Behentrimonium methosulphate, cetyltrimethylammonium chloride, and mixtures thereof.

A particularly useful cationic surfactant for use in conditioners according to the invention is cetyltrimethylammonium chloride, available commercially, for example as GENAMIN CTAC, ex Hoechst Celanese. Another particularly preferred cationic surfactant for use in conditioners according to the invention is behenyltrimethylammonium chloride, available commercially, for example as GENAMIN KDMP, ex Clariant.

Further suitable cationic surfactants include those materials having the CTFA designations Quaternium-5, Quaternium-31, and Quaternium-18. Mixtures of any of the foregoing materials may also be suitable.

Another example of a class of suitable cationic surfactants for use in the invention, either alone or together with one or more other cationic surfactants, is a combination of (i) and (ii) below:

(i) An Amidoamine Corresponding to the General Formula (II):

R¹CONH(CH₂)ₘN(R²)R³ (II)

in which R¹ is a hydrocarbyl chain having 10 or more carbon atoms, R² and R³ are independently selected from hydrocarbyl chains of from 1 to 10 carbon atoms, and m is an integer from 1 to about 10; and

(ii) an acid.

As used herein, the term hydrocarbyl chain means an alkyl or alkenyl chain.

Preferred amidoamine compounds are those corresponding to formula (I) in which

R¹ is a hydrocarbyl residue having from about 11 to about 24 carbon atoms,

R² and R³ are each independently hydrocarbyl residues, preferably alkyl groups, having from 1 to about 4 carbon atoms, and m is an integer from 1 to about 4.

Preferably, R² and R³ are methyl or ethyl groups.

Preferably, m is 2 or 3, i.e. an ethylene or propylene group.

Preferred amidoamines useful herein include stearamido-propyldimethylamine, stearamidopropyldiethylamine, stearamidoethyldiethylamine, stearamidoethyldimethylamine, palmitamidopropyldimethylamine, palmitamidopropyl-diethylamine, palmitamidoethyldiethylamine, palmitamidoethyldimethylamine, behenamidopropyldimethyl-amine, behenamidopropyldiethylmine, behenamidoethyldiethyl-amine, behenamidoethyldimethylamine, arachidamidopropyl-dimethylamine, arachidamidopropyldiethylamine, arachid-amidoethyldiethylamine, arachidamidoethyldimethylamine, and mixtures thereof.

Particularly preferred amidoamines useful herein are stearamidopropyldimethylamine, stearamidoethyldiethylamine, and mixtures thereof.

Commercially available amidoamines useful herein include:

stearamidopropyldimethylamine with tradenames LEXAMINE S-13 available from Inolex (Philadelphia Pa., USA) and AMIDOAMINE MSP available from Nikko (Tokyo, Japan), stearamidoethyldiethylamine with a tradename AMIDOAMINE S available from Nikko, behenamidopropyldimethylamine with a tradename INCROMINE BB available from Croda (North Humberside, England), and various amidoamines with tradenames SCHERCODINE series available from Scher (Clifton N.J., USA).

Acid may be any organic or mineral acid which is capable of protonating the amidoamine in the conditioner composition. Suitable acids useful herein include hydrochloric acid, acetic acid, tartaric acid, fumaric acid, lactic acid, malic acid, succinic acid, and mixtures thereof. Preferably, the acid is selected from the group consisting of acetic acid, tartaric acid, hydrochloric acid, fumaric acid, lactic acid and mixtures thereof.

The primary role of the acid is to protonate the amidoamine in the hair treatment composition thus forming a tertiary amine salt (TAS) in situ in the hair treatment composition. The TAS in effect is a non-permanent quaternary ammonium or pseudo-quaternary ammonium cationic surfactant.

Suitably, the acid is included in a sufficient amount to protonate more than 95 mole % (293 K) of the amidoamine present.

In conditioners for use in the invention, the level of linear cationic conditioning surfactant will generally range from 0.01 to 10%, more preferably 0.05 to 7.5%, most preferably 0.1 to 5% by total weight of cationic conditioning surfactant based on the total weight of the composition.

The Linear Fatty Material (ii)

The composition of the invention comprises from 0.1 to 10 wt % of a linear fatty material.

The combined use of fatty materials and cationic surfactants in conditioning compositions is believed to be especially advantageous, because this leads to the formation of a structured lamellar or liquid crystal phase, in which the cationic surfactant is dispersed.

By “fatty material” is meant a fatty alcohol, an alkoxylated fatty alcohol, a fatty acid or a mixture thereof. Preferably the linear fatty material is selected from a fatty alcohol and a fatty acid, most preferably a fatty alcohol.

Preferably, the alkyl chain of the fatty material is fully saturated. Representative fatty materials comprise from 8 to 22 carbon atoms, more preferably 16 to 22.

Suitable fatty alcohols comprise from 8 to 22 carbon atoms, preferably 16 to 22, most preferably C₁₆ to C₁₈. Fatty alcohols are typically compounds containing straight chain alkyl groups. Preferably, the alkyl groups are saturated. Examples of preferred fatty alcohols include cetyl alcohol, stearyl alcohol and mixtures thereof. The use of these materials is also advantageous in that they contribute to the overall conditioning properties of compositions for use in the invention.

Alkoxylated, (e.g. ethoxylated or propoxylated) fatty alcohols having from about 12 to about 18 carbon atoms in the alkyl chain can be used in place of, or in addition to, the fatty alcohols themselves. Suitable examples include ethylene glycol cetyl ether, polyoxyethylene (2) stearyl ether, polyoxyethylene (4) cetyl ether, and mixtures thereof.

The level of fatty material in conditioners of the invention is suitably from 0.01 to 10, preferably from 0.1 to 10, and more preferably from 0.1 to 5 percent by weight of the total composition. The weight ratio of cationic surfactant to fatty alcohol is suitably from 10:1 to 1:10, preferably from 4:1 to 1:8, optimally from 1:1 to 1:7, for example 1:3.

The Particulate Benefit Agent (iii)

The composition of the invention comprises a particulate benefit agent. The particulate benefit agent is selected from conditioning actives, and mixtures thereof. Preferably, the particulate benefit agent is a conditioning active selected from silicone emulsions, oils and mixtures thereof, most preferably silicone emulsions. More preferably, the conditioning actives are selected from emulsions of dimethicone, dimethiconol, amodimethicone, hydrocarbon oils, fatty esters and mixtures thereof, most preferably, the conditioning actives are selected from emulsions of dimethicone, dimethiconol, amodimethicone, paraffin oil, mineral oil, saturated and unsaturated dodecane, saturated and unsaturated tridecane, saturated and unsaturated tetradecane, saturated and unsaturated pentadecane, saturated and unsaturated hexadecane, polyisobutylene, cocoa butter, palm stearin, sunflower oil, soyabean oil, coconut oil and mixtures thereof.

The following silicones and oils are present in emulsified form in compositions of the invention.

Suitable oils are selected from hydrocarbon oils, fatty esters and mixtures thereof.

Straight chain hydrocarbon oils will preferably contain from about 12 to about 30 carbon atoms. Also suitable are branched chain hydrocarbon oils will preferably contain from about 12 to about 42 carbon atoms. Also suitable are polymeric hydrocarbons of alkenyl monomers, such as C2-C6 alkenyl monomers.

Specific examples of suitable hydrocarbon oils include paraffin oil, mineral oil, saturated and unsaturated dodecane, saturated and unsaturated tridecane, saturated and unsaturated tetradecane, saturated and unsaturated pentadecane, saturated and unsaturated hexadecane, and mixtures thereof. Branched-chain isomers of these compounds, as well as of higher chain length hydrocarbons, can also be used. Another suitable material is polyisobutylene.

Suitable fatty esters are characterised by having at least 10 carbon atoms, and include esters with hydrocarbyl chains derived from fatty acids or alcohols, Monocarboxylic acid esters include esters of alcohols and/or acids of the formula R′COOR in which R′ and R independently denote alkyl or alkenyl radicals and the sum of carbon atoms in R′ and R is at least 10, preferably at least 20. Di- and trialkyl and alkenyl esters of carboxylic acids can also be used.

Particularly preferred fatty esters are mono-, di- and triglycerides, more specifically the mono-, di-, and tri-esters of glycerol and long chain carboxylic acids such as C1-C22 carboxylic acids. Preferred materials include cocoa butter, palm stearin, sunflower oil, soyabean oil and coconut oil.

Preferred silicones are selected from the group consisting of polydimethylsiloxanes and aminofunctionalised silicones, more preferably selected from the group consisting of dimethicone, dimethiconol, amodimethicone and mixtures thereof. Also preferred are blends of aminofunctionalised silicones with dimethicones.

Preferably, the particulate benefit agent is a conditioning active selected from silicone emulsions, oils and mixtures thereof, most preferably silicone emulsions. More preferably, the conditioning actives are selected from emulsions of dimethicone, dimethiconol, amodimethicone, hydrocarbon oils, fatty esters and mixtures thereof, most preferably, the conditioning actives are selected from emulsions of dimethicone, dimethiconol, amodimethicone, paraffin oil, mineral oil, saturated and unsaturated dodecane, saturated and unsaturated tridecane, saturated and unsaturated tetradecane, saturated and unsaturated pentadecane, saturated and unsaturated hexadecane, polyisobutylene, cocoa butter, palm stearin, sunflower oil, soyabean oil, coconut oil and mixtures thereof.

The following silicones and oils are present in emulsified form in compositions of the invention.

Suitable oils are selected from hydrocarbon oils, fatty esters and mixtures thereof.

Straight chain hydrocarbon oils will preferably contain from about 12 to about 30 carbon atoms. Also suitable are branched chain hydrocarbon oils will preferably contain from about 12 to about 42 carbon atoms. Also suitable are polymeric hydrocarbons of alkenyl monomers, such as C2-C6 alkenyl monomers.

Specific examples of suitable hydrocarbon oils include paraffin oil, mineral oil, saturated and unsaturated dodecane, saturated and unsaturated tridecane, saturated and unsaturated tetradecane, saturated and unsaturated pentadecane, saturated and unsaturated hexadecane, and mixtures thereof. Branched-chain isomers of these compounds, as well as of higher chain length hydrocarbons, can also be used. Another suitable material is polyisobutylene.

Suitable fatty esters are characterised by having at least 10 carbon atoms, and include esters with hydrocarbyl chains derived from fatty acids or alcohols, Monocarboxylic acid esters include esters of alcohols and/or acids of the formula R′COOR in which R′ and R independently denote alkyl or alkenyl radicals and the sum of carbon atoms in R′ and R is at least 10, preferably at least 20. Di- and trialkyl and alkenyl esters of carboxylic acids can also be used.

Particularly preferred fatty esters are mono-, di- and triglycerides, more specifically the mono-, di-, and tri-esters of glycerol and long chain carboxylic acids such as C1-C22 carboxylic acids. Preferred materials include cocoa butter, palm stearin, sunflower oil, soyabean oil and coconut oil.

Preferred silicones are selected from the group consisting of polydimethylsiloxanes and aminofunctionalised silicones, more preferably selected from the group consisting of dimethicone, dimethiconol, amodimethicone and mixtures thereof. Also preferred are blends of aminofunctionalised silicones with dimethicones.

Preferred silicone emulsions do not comprise a hydrophobic modification, preferably the silicone emulsion is not a myristyloxyl modified silicone, most preferably not a myristyloxyl modified silicone or a cetyloxyl modified silicone. Most preferably, the silicone emulsions for use in the compositions of the invention are selected from emulsions of dimethicone, dimethiconol, amodimethicone and mixtures thereof.

Suitable silicones include polydimethylsiloxanes which have the CTFA designation dimethicone. Also suitable for use compositions of the invention are polydimethyl siloxanes having hydroxyl end groups, which have the CTFA designation dimethiconol.

Preferably, the silicone is selected from the group consisting of dimethicone, dimethiconol, amodimethicone and mixtures thereof. Also preferred are blends of amino-functionalised silicones with dimethicones.

The viscosity of the emulsified silicone itself (not the emulsion or the final hair conditioning composition) is typically at least 10,000 cst at 25° C. the viscosity of the silicone itself is preferably at least 60,000 cst, most preferably at least 500,000 cst, ideally at least 1,000,000 cst. Preferably the viscosity does not exceed 109 cst for ease of formulation.

Emulsified silicones for use in the compositions of the invention will typically have a D90 silicone droplet size in the composition of less than 30, preferably less than 20, more preferably less than 10 micron, ideally from 0.01 to 1 micron. Silicone emulsions having an average silicone droplet size (D50) of 0.15 micron are generally termed microemulsions.

Silicone particle size may be measured by means of a laser light scattering technique, for example using a 2600D Particle Sizer from Malvern Instruments.

Examples of suitable pre-formed emulsions include Xiameter MEM 1785 and microemulsion DC2-1865 available from Dow Corning. These are emulsions/microemulsions of dimethiconol. Cross-linked silicone gums are also available in a pre-emulsified form, which is advantageous for ease of formulation.

A further preferred class of silicones for inclusion in compositions of the invention are amino functional silicones. By “amino functional silicone” is meant a silicone containing at least one primary, secondary or tertiary amine group, or a quaternary ammonium group. Examples of suitable amino functional silicones include: polysiloxanes having the CTFA designation “amodimethicone”. A preferred amodimethicone is commercially available from Dow Corning as DC 7134.

Specific examples of amino functional silicones suitable for use in the invention are the aminosilicone oils DC2-8220, DC2-8166 and DC2-8566 (all ex Dow Corning).

Suitable quaternary silicone polymers are described in EP-A-0 530 974. A preferred quaternary silicone polymer is K3474, ex Goldschmidt.

Also suitable are emulsions of amino functional silicone oils with non ionic and/or cationic surfactant.

Pre-formed emulsions of amino functional silicone are also available from suppliers of silicone oils such as Dow Corning and General Electric. Specific examples include DC939 Cationic Emulsion and the non-ionic emulsions DC2-7224, DC2-8467, DC2-8177 and DC2-8154 (all ex Dow Corning).

Preferred conditioning actives are selected from the group consisting of polydimethylsiloxanes and aminofunctionalised silicones, blends of aminofunctionalised silicones with dimethicones, hydrocarbon oils, fatty esters and mixtures thereof.

The total amount of particulate benefit agent conditioning active is preferably from 0.1 wt % to 10 wt % of the total composition more preferably from 0.1 wt % to 5 wt %, most preferably 0.25 wt % to 3 wt % is a suitable level.

The Branched Cationic Co-Surfactant (iv)

The composition of the invention comprises a branched cationic co-surfactant.

The branched cationic co-surfactant is selected from structure 1,

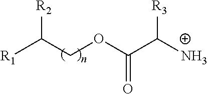

wherein:
R₁ and R₂ comprise linear alkyl chains, saturated or unsaturated, with carbon-carbon chain lengths of from C₄ to C₂₀;
R₃ comprise either a proton or linear or branched alkyl chains, saturated or unsaturated, with carbon-carbon chain lengths of from C₁ to C₅;
n has a range of from 0 to 10;
X is an organic or inorganic anion;

wherein the molar ratio of branched cationic co-surfactant (iv) to linear cationic surfactant (i) is in the range of from 1:20 to 1:1.

R₁ and R₂ comprise linear alkyl chains, saturated or unsaturated, with carbon-carbon chain lengths of from C₄ to C₂₀, preferably from C₆ to C₁₈.

R₃ comprise either a proton or linear or branched alkyl chains, saturated or unsaturated, with carbon-carbon chain lengths of from C₁ to C₅; preferably from C₁ to C₃. Preferably, R₃ is a proton.

n has a range of from 0 to 10, preferably selected from 0 and 1.

The molar ratio of branched cationic co-surfactant (iv) to linear cationic surfactant (i) is in the range of from 1:20 to 1:1, preferably 1:10 to 1:1, most preferably 1:5 to 1:2.

In structure 1, the amine head group is charged within the final formulation. Raw materials include, however, species where the charge is not permanent and can be induced by protonation in the formulation using a strong acid. When R₈ is a proton in the above general formulae therefore, the proton may be present in the raw material or become associated during formulation.

The branched co-surfactant is present in an amount of from 0.01 to 5 wt %, preferably 0.1 to 2, more preferably 0.1 to 1.0, most preferably 0.2 to 0.7 wt % based on the weight of the total composition.

X is an organic or inorganic anion. Preferably, X comprises an anion selected from the halide ions; sulphates of the general formula RSO₃⁻, wherein R is a saturated or unsaturated alkyl radical having 1 to 4 carbon atoms, and anionic radicals of organic acids.

Preferred halide ions are selected from fluoride, chloride, bromide and iodide. Preferred anionic radicals of organic acids are selected from maleate, fumarate, oxalate, tartrate, citrate, lactate and acetate. Preferred sulphates are methanesulphonate and ethanesulphonate.

Most preferably, X⁻ comprises an anion selected from a halide, a methanesulfonate group and an ethanesulphonate group.

An example of a suitable material specific to structure 1 is 2-((2-octyldodecyl)oxy)-2-oxoethan-1-aminium methanesulfonate, which can be synthesized using a modification of the method outlined in Chemistry, A European Journal, 2008, 14, 382. An acid-catalysed condensation reaction of glycine with C₂₀ guerbet alcohol furnishes the desired product in one step.

Composition Rheology

The compositions of the invention provide good viscosity and yield stress properties.

The compositions have a preferred yield stress range of from 30 to 200 Pascals (Pa), most preferably from 40 to 150 Pa peak value at 25° C. and 1 Hz. The method to measure the yield stress uses a serrated parallel-plate geometry, 40 mm in diameter, attached to a suitable rheometer capable of applying oscillations at a constant frequency of 1 Hz, and an amplitude sweep in the range of 0.1% to 2000%. The amplitude sweep range is applied at no more than ten points per decade of strain range covered at no more than 4 cycles per amplitude. The instrument should be operated under controlled strain, such as with the ARES G2 Rheometer from TA Instruments. The geometry's temperature should be set at 25° C. by means of, for example, a Peltier-controlled plate, or a recirculating bath. The yield stress is determined by plotting the elastic stress against strain amplitude, and at the peak of the curve, the maximum value is quoted as the yield stress. The elastic stress is calculated as the multiplication of (storage modulus)*(strain amplitude), each readily obtained from the instrument.

The compositions have a viscosity of from 5,000 to 750,000 centipoise, preferably from 50,000 to 600,000 centipoise, more preferably from 50,000 to 450,000 as measured at 30° C. on a Brookfield RVT using a Spindle A or B at 0.5 rpm for 60 seconds on a Helipath stand.

A preferred conditioner comprises a conditioning gel phase. These conditioners have little or no vesicle content. Such conditioners and methods for making them are described in WO2014/016354, WO2014/016353, WO2012/016352 and WO2014/016351.

A composition comprising such a conditioning gel phase confers a Draw Mass of from 1 to 250 g, preferably 2 to 100 g, more preferably 2 to 50 g, even more preferably 5 to 40 g and most preferably 5 to 25 g to hair treated with the composition.

Draw Mass is the mass required to draw a hair switch through a comb or brush. Thus the more tangled the hair the greater the mass required to pull the switch through the comb or brush, and the greater the level of condition of the hair, the lower the Draw Mass.

The Draw Mass is the mass required to draw a hair switch, for example of weight 1 to 20 g, length 10 to 30 cm, and width 0.5 to 5 cm through a comb or brush, as measured by first placing the hair switch onto the comb or brush, such that from 5 to 20 cm of hair is left hanging at the glued end of the switch, and then adding weights to the hanging end until the switch falls through the comb or brush.

Preferably, the hair switch is of weight 1 to 20 g, more preferably 2 to 15 g, most preferably from 5 to 10 g. Preferably, the hair switch has a length of from 10 to 40 cm, more preferably from 10 to 30 cm, and a width of from 0.5 to 5 cm, more preferably from 1.5 to 4 cm.

Most preferably, the Draw Mass is the mass required to draw a hair switch, for example of weight 10 g, length 20 cm, and width 3 cm through a comb or brush, as measured by first placing the hair switch onto the comb or brush, such that from 20 cm of hair is left hanging at the glued end of the switch, and then adding weights to the hanging end until the switch falls through the comb or brush.

Further Ingredients

The composition according to the invention may comprise any of a number of ingredients which are common to hair conditioning compositions.

Other ingredients may include, preservatives, colouring agents, polyols such as glycerine and polypropylene glycol, chelating agents such as EDTA, antioxidants such as vitamin E acetate, fragrances, antimicrobials and sunscreens. Each of these ingredients will be present in an amount effective to accomplish its purpose. Generally these optional ingredients are included individually at a level of up to about 5% by weight of the total composition.

Preferably, the further ingredients include perfumes, preservatives, colours and conditioning silicones.

Mixtures of any of the above active ingredients may also be used.

Generally, such ingredients are included individually at a level of up to 2%, preferably up to 1%, by weight of the total composition.

The compositions of the invention are preferably free from thickening agents for example thickening polymers. Examples of thickening polymers include polyquaternium thickeners (such as polyquaternium-10, polyquaternium-39); guar based thickeners (such as guar hydroxy ammonium chloride); Polyethylene Glycol (PEG) based thickeners (such as PEG 90M, PEG 14M, PEG 150 distearate) and the like.

Embodiments of the invention are given in the following examples, in which all percentages are quoted by weight based on total weight unless otherwise stated.

### EXAMPLES

### Example 1: Composition 1 in Accordance with the Invention and Comparative Composition A

The following compositions were prepared:

TABLE 1
Compositions of example A (comparative) and
example 1 (in accordance with the invention).

| | | Example 2 |
|---|---|---|
| | | Gel phase |
| | | including branched |
| | Example A | glycine ester |
| Ingredient | Comparative | (Structure 1) |
| Behentrimonium Chloride | 2.00 | 1.60 |
| Cetearyl Alcohol | 4.00 | 3.20 |
| 2-((2-octyldodecyl)oxy)-2-oxoethan- | — | 0.5 |
| 1-aminium methanesulfonate | | |
| Dimethicone 600K and | 1.00 | 1.00 |
| Amodimethicone 2000 nm | | |
| Disodium EDTA | 0.05 | 0.05 |
| Water | to 100 | to 100 |

The conditioners in examples A and 1 were prepared using the following method:
1. Surfactants and fatty materials were added to a suitable vessel and heated to above the melting point of the fatty materials.
2. The molten blend was added to a suitable amount of water according to the compositions in Table 1, at a temperature of between room temperature and below the melting point of the fatty materials.
3. The mixture was mixed until opaque and thick.
4. The heat was then turned off, cooled to room temperature, and the rest of the water was added along with the remaining materials.
5. Finally, the formulation was mixed at high shear using a suitable homogenising device.

Predicted conditioning performance (silicone deposition by XRF) is given in Table 2, which is given below:

| | | Example 1 |
|---|---|---|
| | | Gel phase |
| | Example A | including branched |
| | Comparative | glycine ester |
| Conditioning | No | Significant |
| performance | advantage | advantage |

1. A conditioning composition comprising:
(i) 0.01 to 10 wt % of a linear cationic conditioning primary surfactant; selected from structure 1 and mixtures thereof: 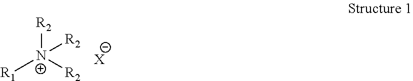
wherein:
R¹ comprises a linear alkyl chain having a carbon-carbon chain length of from C₁₆ to C₂₄;
R² comprises a proton or a linear alkyl chain having a carbon-carbon chain length of from C₁ to C₄, or a benzyl group; and
X is an organic or inorganic anion;
(ii) 0.1 to 10 wt % of a linear fatty material;
(iii) a particulate benefit agent selected from conditioning actives and mixtures thereof;
(iv) 0.01 to 5 wt % of a linear di-alkyl cationic co-surfactant, selected from structure 2 and mixtures thereof 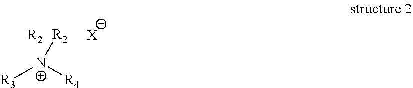
wherein:
R² comprises a proton or a linear alkyl chain having a carbon-carbon chain length of from C₁ to C₄, or a benzyl group;
R³ comprises a linear alkyl chain having a carbon-carbon chain length of from C₃ up to but not including C₁₆;
R⁴ comprises linear alkyl chain having a carbon-carbon chain length of from C₃ to C₂₄; and
X is an organic or inorganic anion;
wherein the carbon-carbon chain length of R₁ in structure 1 differs from the carbon-carbon chain length of R₃ in structure 2 by at least 3 carbon atoms, such that the carbon-carbon chain length of R₁ in structure 1 is longer than the carbon-carbon chain length of R₃ in structure 2; and
wherein the molar ratios of linear dialkyl cationic co-surfactant (iv) to linear cationic conditioning primary surfactant (i) are in the range of from 1:20 to 1:1.

2. The conditioning composition as claimed in claim 1, wherein the carbon-carbon chain length of R₁ in structure 1 differs from the carbon-carbon chain length of R₃ in structure 2 by from 3 to 12 carbon atoms, such that the carbon-carbon chain length of R₁ in structure 1 is longer than the carbon-carbon chain length of R₃ in structure 2.

3. The conditioning composition as claimed in claim 1, wherein R₃ comprises a linear alkyl chain having a carbon-carbon chain length of from C₃ to C₁₄.

4. The conditioning composition as claimed in claim 1, wherein in Structure 2, the R₄ group is the same as the R₃ group.

5. The conditioning composition as claimed in claim 4, wherein in Structure 2, the R₄ group and the R₃ group have a carbon-carbon chain length of from C₁₀ to C₁₄.

6. The conditioning composition as claimed in claim 1, wherein the linear cationic conditioning surfactant is selected from behenyltrimethylammonium chloride, behentrimonium methosulphate, cetyltrimethylammonium chloride, and mixtures thereof.

7. The conditioning composition as claimed in claim 1, wherein the conditioning actives are selected from silicone emulsions and oils.

8. The composition as claimed in claim 7, wherein the conditioning actives are selected from emulsions of dimethicone, dimethiconol, amodimethicone, hydrocarbon oils, fatty esters and mixtures thereof.

9. The composition as claimed in claim 8, wherein the conditioning actives are selected from emulsions of dimethicone, dimethiconol, amodimethicone, paraffin oil, mineral oil, saturated and unsaturated dodecane, saturated and unsaturated tridecane, saturated and unsaturated tetradecane, saturated and unsaturated pentadecane, saturated and unsaturated hexadecane, polyisobutylene, cocoa butter, palm stearin, sunflower oil, soyabean oil, coconut oil and mixtures thereof.

10. The composition as claimed in claim 8, wherein the conditioning actives are selected from emulsions of dimethicone, dimethiconol, amodimethicone and mixtures thereof.

11. The conditioning composition as claimed in claim 1, wherein the particulate benefit agent conditioning active is present in an amount of from 0.1 wt % to 10 wt % of the total composition.

12. The conditioning composition as claimed in claim 1, wherein the linear dialkyl cationic co-surfactant is present in an amount of from 0.1 to 2 wt %, preferably 0.2 to 0.7 wt %.

13. The conditioning composition as claimed in claim 1, wherein the molar ratios of linear dialkyl cationic co-surfactants (iv) to linear cationic surfactants (i) are in the range of from 1:10 to 1:1.

14. The conditioning composition as claimed in claim 1, which has a viscosity of from 5,000 to 750,000 centipoise, as measured at 30° C. on a Brookfield RVT using a Spindle A or B at 0.5 rpm for 60 seconds on a Helipath stand.

15. A method of increasing deposition of a particulate benefit agent selected from conditioning actives and mixtures thereof to hair comprising:
applying to hair a conditioning composition as claimed in claim 1, and
rinsing the hair with water.

16. The conditioning composition as claimed in claim 1, wherein in structure 1, R₁ comprises a linear alkyl chain having a carbon-carbon chain length of from Cis to C₂₂; and R₂ comprises a proton or a linear alkyl chain having a carbon-carbon chain length of from C₁ to C₂.

17. The conditioning composition as claimed in claim 1, wherein in structure 2, R₂ comprises a proton or a linear alkyl chain having a carbon-carbon chain length of from C₁ to C₂; R₃ comprises a linear alkyl chain having a carbon-carbon chain length of from C₁₀ to C₁₄; and R₄ comprises linear alkyl chain having a carbon-carbon chain length of from Cm to C₁₄.

18. The conditioning composition as claimed in claim 2, wherein the carbon-carbon chain length of R₁ in structure 1 differs from the carbon-carbon chain length of R₃ in structure 2 by from 6 to 10 carbon atoms, such that the carbon-carbon chain length of R₁ in structure 1 is longer than the carbon-carbon chain length of R₃ in structure 2.

19. The conditioning composition as claimed in claim 11, wherein the particulate benefit agent conditioning active is present in an amount of from 0.1 wt % to 5 wt % of the total composition.

20. The conditioning composition as claimed in claim 11, wherein the particulate benefit agent conditioning active is present in an amount of from 0.25 wt % to 3 wt % of the total composition.

21. The conditioning composition as claimed in claim 12, wherein the linear dialkyl cationic co-surfactant is present in an amount of from 0.2 to 0.7 wt %.

22. The conditioning composition as claimed in claim 13, wherein the molar ratios of linear dialkyl cationic co-surfactants (iv) to linear cationic surfactants (i) are in the range of from 1:5 to 1:2.
